(19) United States
(12) Patent Application Publication
(10) Pub. No.: US 2023/0410966 A1
(43) Pub. Date: Dec. 21, 2023

(54) INTELLIGENT MEDICAL RESOURCE SHARING PLATFORM

(71) Applicant: YI-YUN WANG, NEW TAIPEI CITY (TW)

(72) Inventor: Yi-Yun WANG, NEW TAIPEI CITY (TW)

(21) Appl. No.: 17/822,142

(22) Filed: Aug. 25, 2022

(30) Foreign Application Priority Data
Jun. 15, 2022 (TW) 111122113

(51) Int. Cl. G16H 10/65; G06Q 30/00; G16H 15/00; G16H 50/30

(52) U.S. Cl. CPC G16H 10/65; G06Q 30/018; G16H 15/00; G16H 50/30

(57) ABSTRACT

An intelligent medical resource sharing platform is provided. The intelligent medical resource sharing platform includes a server, at least one user end device, at least one physician end device, and a consultation platform. The server includes a physiological value database, a personal record database, and a physiological value processing module. The physiological value processing module is correspondingly coupled to the physiological value database and the personal record database, and processes physiological data transmitted from the physiological value database and the personal record database.

### CROSS-REFERENCE TO RELATED PATENT APPLICATION

This application claims the benefit of priority to Taiwan Patent Application No. 111122113, filed on Jun. 15, 2022. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a medical resource sharing platform, and more particularly to a sharing platform which integrates medical resources and advise a user on medical information.

### BACKGROUND OF THE DISCLOSURE

Conventionally, patients are often unable to determine which physician they need to consult after the medical examination or after receiving the health examination report, resulting in certain patients not being followed up and causing potential health concerns for patients. In addition, with the epidemic pandemic, the reluctance of patients to go to the hospital has increased, making the health of patients insecure, which needs to be improved.

### SUMMARY OF THE DISCLOSURE

In response to the above-referenced technical inadequacies, the present disclosure provides an intelligent medical resource sharing platform, which integrates a patient end, a physician end, and a pharmacist end, so that the patient can upload a health state or physiological data. In addition, through remote consultation with the physician, the patient can clearly understand how to effectively improve symptoms. In addition, through cooperating with the pharmacist who is at a remote location to dispense medicines, the patient can get relevant medicines to treat his/her conditions. The present disclosure also enable the user to evaluate his/her psychological state, metabolic state, endocrinal state, and whether the user is in a sub-healthy state in advance by inputting relevant data in advance through the intelligent medical resource sharing platform of the present disclosure. Further, it is also possible to further evaluate and recommend the user on whether to consult practitioners of traditional Chinese medicine or physicians of Western medicine.

In one aspect, the present disclosure provides an intelligent medical resource sharing platform, which includes a server, at least one user end device, and at least one physician end device. The server includes a physiological value database, a personal record database, a physiological value processing module, a physician database, a user credentials real-time confirmation module, and a physician credentials real-time confirmation module. The physiological value processing module is correspondingly coupled to the physiological value database and the personal record database, and processes physiological data transmitted from the physiological value database and the personal record database. The user credentials real-time confirmation module is coupled to the server and used to perform checks and confirm an identity of a user. The physician credentials real-time confirmation module is coupled to the server and used to perform checks and validate a certification of a physician. The at least one user end device is wirelessly connected to the server. The at least one user end device includes a user identity input module, a user real-time credentials check reply module, and a user physiological data input module. The user real-time credentials check reply module is coupled to the user identity input module and used for real-time reception and reply of a message transmitted by the user credentials real-time confirmation module. The user physiological data input module is coupled to the user real-time credentials check reply module and used to upload the physiological data of the user to the personal record database. The at least one physician end device is wirelessly connected to the server. The at least one physician end device includes a physician credentials input module and a physician real-time credentials check reply module. The physician credentials input module provides a physician to perform entry of individual physician credentials. The physician real-time credentials check reply module is coupled to the physician credentials input module and used for real-time reception and reply of a message transmitted by the physician credentials real-time confirmation module. The at least one user end device is used by at least one user, and the at least one user uses the intelligent medical resource sharing platform to obtain a medical department recommendation.

In certain embodiments, the physiological value processing module includes a data receiving unit, a data comparison unit, and a data evaluation unit. The data receiving unit is used to receive the physiological data uploaded by the user physiological data input module. The data comparison unit is coupled to the data receiving unit and used to compare data uploaded by the user physiological data input module to the personal record database with data in the physiological value database. The data evaluation unit is coupled to the data receiving unit. The data evaluation unit evaluates a physiological state of the user according to the physiological data analyzed by the data comparison unit so as to generate a physiological data analysis report and transmit the physiological data analysis report to the user end device and the physician end device, correspondingly.

One of the beneficial effects of the present disclosure is that, the intelligent medical resource sharing platform provided by the present disclosure enables consultation between the physician, the pharmacist, and the user through a consultation platform, which is not limited by distance or time. Through generating the physiological data analysis report by the physiological value processing module, an uncertainty of the user in seeking medical consultation can be effectively reduced. The user often goes to the hospital only because of self-doubt. By comparing the physiological data in the physiological value database, the physiological data analysis report is generated, which allows the user to understand his/her health state before consulting the physician. In addition, through the evaluation and recommendation provided by the intelligent medical resource sharing platform of the present disclosure, the user can have a clearer understanding of which medical department should choose, so that a waste of medical resources can be effectively reduced. Further, when the user really needs to consult the physician, a remote consultation with the physician and the pharmacist can be conducted through the consultation platform, so that the consultation can be kept confidential. In addition, the user can avoid a risk of infection outside of the home. Even if the user is located in a remote site, sufficient medical resources are also available.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of an architecture of an intelligent medical resource sharing platform according to an embodiment of the present disclosure;

FIG. 2 is a schematic view of a user end device which provides a suggested department of consultation according to an embodiment of the present disclosure;

FIG. 3 is a schematic view of a coupling arrangement of a server according to an embodiment of the present disclosure;

FIG. 4 is a schematic view of a coupling arrangement of hardware circuits between a calculator and a data evaluation unit according to an embodiment of the present disclosure;

FIG. 5 is a schematic view of a coupling arrangement of a wireless communication between the server and a user end, a physician end device and a pharmacist end device according to an embodiment of the present disclosure;

FIG. 6 is a schematic view of a physiological data analysis report according to an embodiment of the present disclosure;

FIG. 7 is a schematic view of the physiological data analysis report according to another embodiment of the present disclosure; and

FIG. 8 is a schematic view of the physiological data analysis report according to yet another embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### EMBODIMENTS

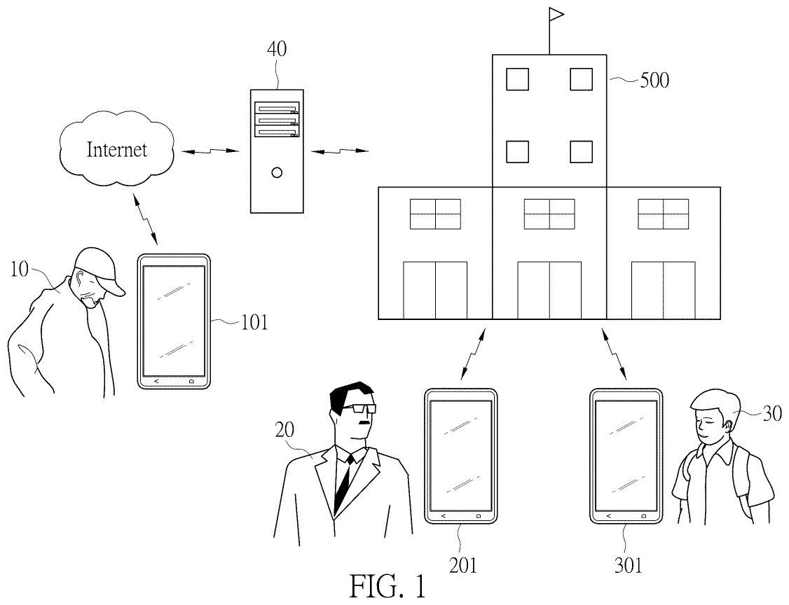

Referring to FIG. 1, FIG. 1 is a schematic view of an architecture of an intelligent medical resource sharing platform according to an embodiment of the present disclosure. The intelligent medical resource sharing platform of the present disclosure includes a server 40, a user end device 101, a physician end device 201, a pharmacist end device 301, and a consultation platform 500. As shown in FIG. 1, the server 40 is correspondingly connected to the user end device 101, the physician end device 201, and the pharmacist end device 301, mainly in a wireless manner. However, in order to match various connection designs of practical connections between the user end device 101, the physician end device 201, and the pharmacist end device 301, the present disclosure is not limited thereto. On the other hand, one user 10, one physician 20, one pharmacist 30 and one of the consultation platform 500 are exemplarily shown in FIG. 1. However, in practical application, the intelligent medical resource sharing platform of the present disclosure can be provided to multiple users 10, multiple physicians 20, multiple pharmacists 30, and multiple of the consultation platforms 500 at the same time. That is, it should be noted that the intelligent medical resource sharing platform of the present disclosure is not limited to the user by one person, but can be used by more than one person at the same time.

It should be noted that, in another embodiment of the present disclosure, the consultation platform 500 may be omitted from the intelligent medical resource sharing platform of the present disclosure. That is, the user 10, the physician 20, and the pharmacist 30 can correspondingly and directly connected to the server 40 in the wireless manner so as to perform an operation of the intelligent medical resource sharing platform. In this way, the present disclosure can also be realized to reduce a waste of medical resources. In addition, a remote consultation with the physician and the pharmacist can also be conducted, which ensures privacy during consultation. Moreover, the user can avoid a risk of infection outside of the home. Even if the user is located in a remote site, sufficient medical resources are also available. At this time, the server 40 is large or huge server hardware.

As shown in FIG. 1, the consultation platform 500 can be correspondingly coupled to the server 40, the user end device 101, the physician end device 201, and the pharmacist end device 301. The coupling refers to a practical connection by wireless communication, including but not limited to 4G communication, 5G communication, Wi-Fi® communication, Li-Fi communication, or satellite communication. Further, when any two of the user end device 101, the physician end device 201, and the pharmacist end device 301 are connected to the server 40 (e.g., connection through wireless communication), any two of the user 10, the physician 20, and the pharmacist 30 can virtually meet each other in the consultation platform 500 through a virtual reality (VR) system in the corresponding devices (e.g., the user end device 101, the physician end device 201, and the pharmacist end device 301). In addition, the user end device 101, the physician end device 201, and the pharmacist end device 301 are not limited to using the VR system, but may also an augmented reality (AR) system or a hybrid reality (XR) system. Therefore, the physician 20, the pharmacist 30, and the user 10 can conduct consultation through a metaverse in the consultation platform 500. In this way, the medical resources can be free from distance. In addition, the physician 20, the pharmacist 30, and the user 10 can effectively communicate with each other, and the risk of infection outside of the home can be avoided.

On the other hand, data transmitted between the server 40, the user end device 101, the physician end device 201, the pharmacist end device 301, and the consultation platform 500 as shown in FIG. 1, is further encrypted by using blockchain technology. Therefore, the consultation conducted between the physician 20, the pharmacist 30, and the user 10 can be kept confidential, and conversation between the physician 20, the pharmacist 30, and the user 10 can also be recorded, so that rights and interests of the healthcare providers and the patient can be protected.

Referring further to FIG. 1, the server 40 as shown in FIG. 1 can be installed in the consultation platform 500. That is, in practice, if the consultation platform 500 is a large teaching hospital or a large urban hospital, a large one of the server 40 can be built in so as to store and process a large amount of user related physiological data since such large hospitals have more hardware resources. On the other hand, if the consultation platform 500 is a community medical clinic or a rural hospital, such hospitals do not have enough hardware resources. In this case, the large amount of user related physiological data is stored and processed by the server 40 in a remote site (may be located in a giant server room in a certain region). At this time, the community medical clinic (i.e., another type of the consultation platform 500) is connected the server 40 in the remote site through wireless communication, so as to perform an operation of intelligently sharing the medical resources.

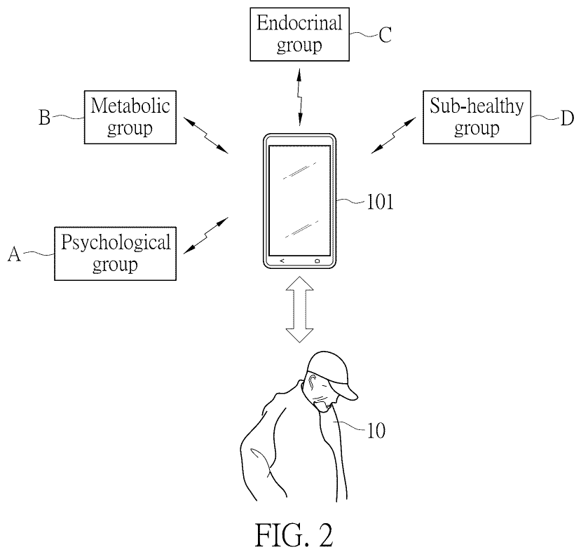

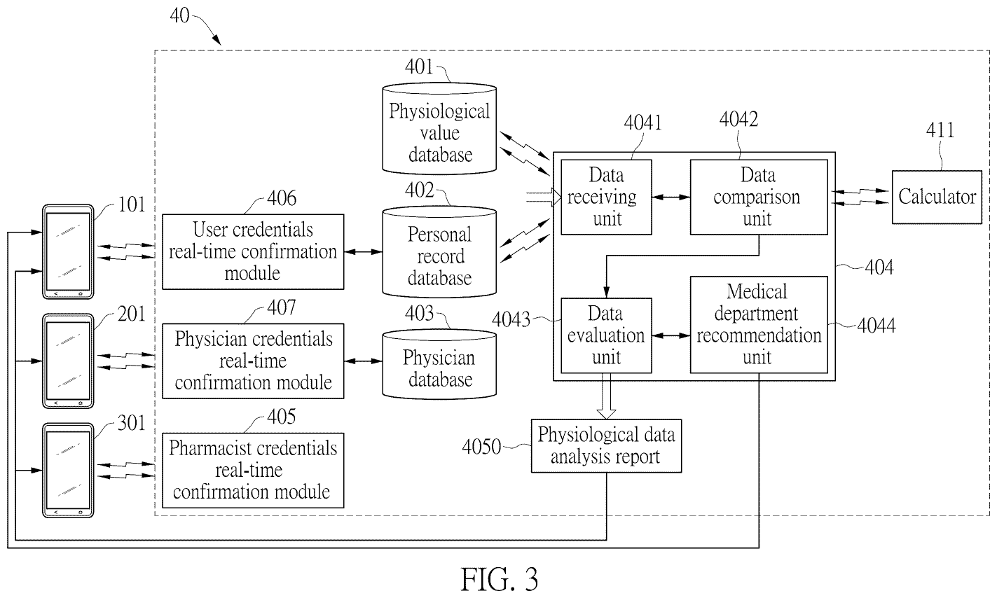

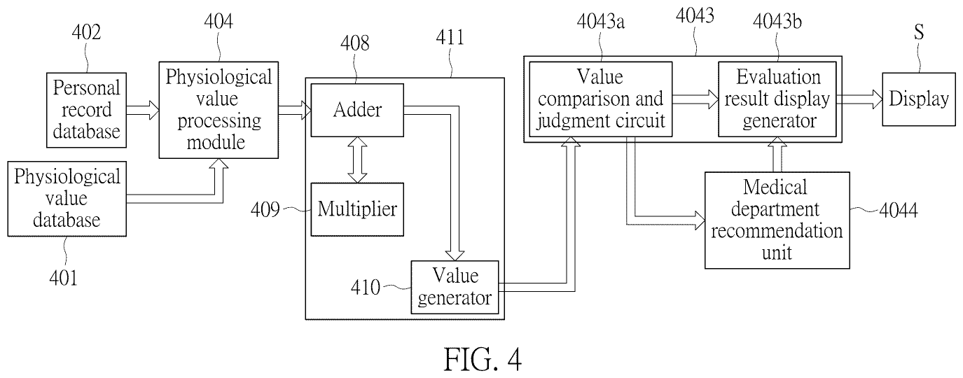

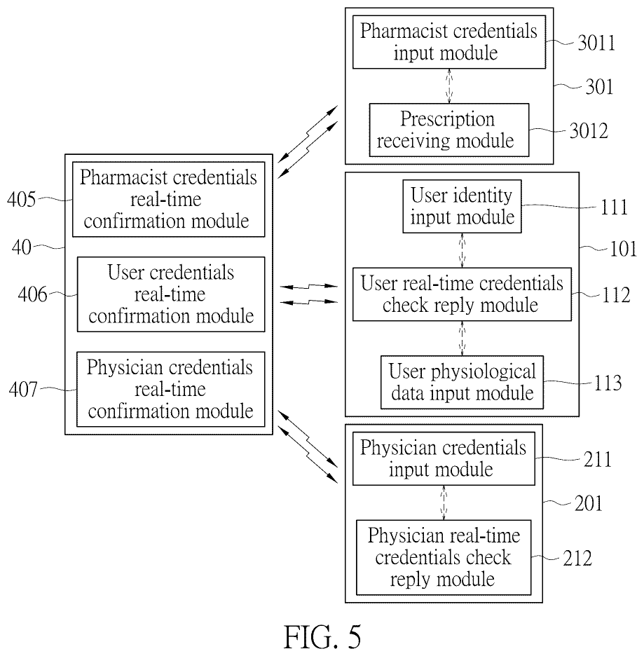

Referring to FIG. 2, the user 10 can further differentiate the related physiological data of the user 10 by using a user physiological data input module 113 (referring to FIG. 5 for the user physiological data input module 113, which will be further described in the following) specially disposed in the user end device 101. In one particular embodiment of the present disclosure, the related physiological data of the user 10 can be divided into groups including a psychological group A, a metabolic group B, an endocrinal group C, and a sub-healthy group D. The four groups described above refer to one embodiment of the present disclosure only, and for platform developers, they are not limited by the four groups described above. On the other hand, results calculated or evaluated by such different groups of the related physiological data are further transmitted to a personal record database 402 (referring to FIG. 3 for the personal record database 402) in the server 40 for storage. With reference to FIG. 3 and FIG. 4, a detailed description for the psychological group A, the metabolic group B, the endocrinal group C, and the sub-healthy group D is provided. That is, the hardware circuit architecture as shown in FIG. 3 and FIG. 4 is to realize the operation of intelligently sharing the medical resources among different groups such as the psychological group A, the metabolic group B, the endocrinal group C, and the sub-healthy group D as shown in FIG. 2.

Referring to FIG. 3, the server 40 includes a physiological value database 401, the personal record database 402, a physiological value processing module 404, a physician database 403, a user credentials real-time confirmation module 406, a physician credentials real-time confirmation module 407, a pharmacist credentials real-time confirmation module 405, and a calculator 411 that is coupled to the physiological value processing module 404. The physiological value processing module 404 outputs a physiological data analysis report 4050. Further, the physiological value processing module 404 includes a data receiving unit 4041, a data comparison unit 4042, a data evaluation unit 4043, and a medical department recommendation unit 4044. The physiological value database 401 is used to store standard health data of the user 10 or generally accepted standard health data. The standard health data can also be taken from standard data of the relevant governmental or non-governmental units, or can be standardized according to a self-defined health data of the user 10. An internal composition of the physiological value database 401 is further divided into a psychological physiological data group, a metabolic physiological data group, a traditional Chinese medicine physiological data group, and a sub-healthy physiological data group to correspond to the different groups as shown in FIG. 2, so as to generate a corresponding and classified operation. In addition, the personal record database 402 is used to store personal physiological data of the user 10 that is wirelessly transmitted to the server 40. Similarly, contents of the personal physiological data can also be divided into a psychological personal record group, a metabolic personal record group, an endocrinal personal record group, and a sub-healthy personal record group. Further, the personal physiological data recorded includes, but not limited to, psychological status assessment, personal metabolic data, basic personal physiological data (e.g. height, weight, body fat percentage, skeletal muscle ratio, heart rate variability, blood oxygen level, etc.), personal endocrine status, sub-health self-assessment results, and genetic test results. Sub-health refers to a state in which a person is between health and disease. Factors affecting sub-health depend on genetic influences, environmental pollution, stressful life, physical and mental pressure, bad habits, excessive work, etc. In addition, the user 10 can also upload the physiological data through a default questionnaire in the user end device 101. The physician database 403 is used to store personal information of licensed physicians, including information on physicians of various disciplines and specialties and their corresponding profiles. The physician database 403 can further store information such as relevant experience recorded in a course of each physician's practicing career and professional publications.

Referring to FIG. 3, the server 40 is individually connected to the user end device 101, the physician end device 201, and the pharmacist end device 301 by wireless communication in a two-way manner. The user end device 101 is wirelessly connected to the user credentials real-time confirmation module 406, so as to serve as an identity verification and authentication portal of the intelligent medical resource sharing platform of the present disclosure for the user 10. The physician end device 201 is wirelessly connected to the physician credentials real-time confirmation module 407 in the two-way manner, so as to serve as an identity verification and authentication for the professional physicians who use the intelligent medical resource sharing platform of the present disclosure, and even serve as authentication of a professional field, a professional classification, or a cross-disciplinary professional field of the professional physicians, so that more medical professional services are provided in the intelligent medical resource sharing platform of the present disclosure. The pharmacist end device 301 is wirelessly connected to the pharmacist credentials real-time confirmation module 405 in the two-way manner, so as to serve as an identity verification and authentication for the pharmacist 30.

As shown in FIG. 3, the user credentials real-time confirmation module 406 is bidirectionally coupled to the personal record database 402, which is used to record and store personal data or physiological date of the user 10, including previously established data or current real-time physiological data, for later calculation and comparison. Similarly, the physician credentials real-time confirmation module 407 is bidirectionally coupled to the physician database 403, which is used to store personal data of the physician 20, who has entered or used the intelligent medical resource sharing platform of the present disclosure, including data previously established or stored in advance, as well as current real-time physiological data.

Further, as shown in FIG. 3, the physiological value processing module 404 is bidirectionally coupled to the physiological value database 401. Although FIG. 3 shows that the physiological value processing module 404 and the physiological value database 401 wirelessly transmit to each other, in practice, such connection may be wireless, wired, or implemented by direct wiring on a printed circuit board (PCB). In one embodiment, in a case of wireless transmission, the physiological value database 401 is set up independently and is placed on a different building at a remote location. At this time, the physiological value database 401 is communicatively coupled to the physiological value processing module 404 in the server 40 in the wireless manner. Similarly, a connection between the personal record database 402 and the physiological value processing module 404 can be wired, wireless, or directly implemented on the PCB.

As shown in FIG. 3, after the data receiving unit 4041 in the physiological value processing module 404 receives the relevant data from the physiological value database 401 and the personal record database 402, the data receiving unit 4041 transmits the related physiological data and the personal data of the user 10 to the data comparison unit 4042. The data comparison unit 4042 compares the personal data of the user 10 with basic or reference data stored in the physiological value database 401, or even quantifies such data for comparison, and then performs calculations and operations so as to obtain a comparison result that is calculated. In practice, the calculator 411 is used for further numerical calculation so as to obtain the comparison result that is calculated. The comparison result is then transmitted to the data evaluation unit 4043, that is, with the comparison result, further data evaluation and prediction can be performed, and an evaluation result can be transmitted to the medical department recommendation unit 4044. At this time, the medical department recommendation unit 4044 can further transmit a recommend medical department to the user end device 101 of the user 10, so that the user 10 can obtain a useful reference for the recommended medical department, and finally the user 10 can decide which medical department to consult personally. On the other hand, the evaluation result transmitted by the data evaluation unit 4043 is also transmitted with the physiological data analysis report 4050. The physiological data analysis report 4050 is then transmitted to the user end device 101, the physician end device 201, and the pharmacist end device 301 for reference by the user 10, the physician 20, and the pharmacist 30, respectively.

In order to achieve the goal of present disclosure of enabling the user to understand his/her own health status in advance and providing relevant evaluations and recommendations, the present disclosure is to numerically process the personal physiological data recorded by the user 10 in advance, and then to perform logical formula operations, so as to obtain useful evaluations and recommendations.

First of all, in one particular embodiment of the present disclosure, for the evaluation of the psychological group A, a psychological state questionnaire (in practice, it may be a depression scale) is preset in the user end device 101 for evaluating a depression tendency of the user 10. It can be set that if the user scores more than 29, he/she has high depression tendency; if the user 10 scores between 19 and 28, he/she has moderate depression tendency; if the user scores between 15 and 18, he/she has low depression tendency; if the user 10 scores between 9 and 15, he/she has occasional stress; if the user 10 scores less than 8, he/she is in good physical and mental health. As for an origin of the aforementioned psychological state score value, it is described as follows.

The implementation of the aforementioned depression scale can be based on a self-rating given by the user 10, with a basic score of 1 point for each item, but with different weightings for different items. The depression score can be obtained according to the following questions, such as E1: I feel like crying; E2: I feel bad; E3: I feel more easily angry than before; E4: I do not sleep well; E5: I feel like I do not want to eat; E6: I feel stuffy in my chest (chest tightness); E7: I feel uncomfortable (unpleasant); and E8: I feel tired and weak (physically weak, lacking of energy and physical strength). The weighting of each of the above questions is 1. In addition, the depression score can be obtained according to the following questions, such as F1: I feel annoyed; F2: I feel my memory is bad; F3: I feel that I cannot concentrate on my work; and F4: I feel that I think about things or do things more slowly than usual. The weight of each of the above questions is 1.5. Further, the depression score can be obtained according to the following questions, such as G1: I feel less confident than before; G2: I feel more likely to think in a bad way; G3: I feel that I take things too hard, or even I want to die; G4: I feel disinterested in everything; G5: I feel unwell (e.g., headache, dizziness, palpitations, and upset stomach); and G6: I feel useless. The weight of each of the above questions is 2. Each of the above items is an independent question, and the user 10 is given the score for the item when the user 10 meets the characteristics of the item or meets the situation at hand.

The present disclosure provides a parameter H=E+1.5F+2G; where E=E1+E2+E3+E4+E5+E6+E7+E8, F=F1+F2+F3+F4, and G=G1+G2+G3+G4+G5+G6. The H parameter can be regarded as a parameter for evaluating the psychological state of the user 10.

For example, if the user 10 has the idea of crying, the user 10 is given 1 point, and if not, the user 10 is given 0 point. Or if the user 10 has the idea of taking things too hard or even dying, the user 10 is given 2 points, and if not, the user 10 is given 0 point. According to a sum of the scores for each item, the user 10 can obtain the score on the depression scale. In this way, the embodiments of the present disclosure also correspond to the aforementioned numerical processing and related operations, and serve as a reference for subsequent psychological evaluation and consultation recommendations.

In addition, the preset sub-health questionnaire in the user end device 101 is used by the user 10 to make a judgment according to the current situation. For example, when the score obtained by the user 10 (a parameter for such the score is set to be R) exceeds 16 points, the user 10 is determined to be in a sub-healthy state. The score of each question is 1 point, but different questions have different weightings according to the content of the embodiments of the present disclosure.

Questions with a weighting of 1 in the sub-health questionnaire include A1: when I wake up in the morning, my hair often falls out; A2: I feel a bit depressed and stare out of the window; A3: I am afraid to walk into the office and find my work tiresome; A4: I do not want to face colleagues and supervisors, and I have a tendency of autism; A5: My work efficiency has dropped, and my supervisors are dissatisfied with me; A6: I cannot get high emotionally at work, and more curiously, I often feel irritable but have no energy to vent; A7: I have no appetite; A8: I feel tired of work and want to go home as soon as possible to rest; A9: I am very sensitive to the pollution and noise, and I long for peace; A10: I feel that my immune system is weakened and I am susceptible to the flu; and A11: I feel not as keen on gatherings with friends as before. The user who meets the condition of each of A1 to A11 is given 1 point, and if not, the user is given 0 point. Accordingly, a parameter PA=A1+A2+A3+A4+A5+A6+A7+A8+A9+A10+A11 is provided.

The embodiments of the present disclosure specifically provides questions that require additional weighting for the sub-health questionnaire, including B1: I have trouble with my short-term memory; B2: I feel tired and chest tightness after working for a short time; B3: I feel bad sleep quality; B4: I feel that there is a clear trend of weight loss; and B5: I feel that my sexual ability is decreased and I have no desire for sex. Accordingly, a parameter PB=B1+B2+B3+B4+B5 is provided. In addition, the parameter R=PA+(3×PB) (Equation 1) is provided. That is, in particular, in the embodiment of the present disclosure, the weighting of each of B1 to B5 is 3. Certainly, in practice, different platform developers can adjust different weightings according to different platform attributes to meet practical requirements. According to the above, the parameter R can be further used to judge whether or not the user 10 is in the sub-healthy state.

Each of the above items is an independent question, and the user 10 is given the score for the item when the user 10 meets the characteristics of the item or meets the situation at hand. For example, if the user 10 has the hair loss problem in the morning, the user 10 is given 1 point, and if not, the user 10 is given 0 point. Or if the user 10 has a reduced sexual ability, the user 10 is given 2 points, and if not, the user 10 is given 0 point. According to a sum of the scores for each item, the user 10 can obtain the score on the sub-health questionnaire.

When the user 10 completes answering the preset questions, the questionnaire results are transmitted to the calculator 411 through the user end device 101, and a value generator 410 calculates a survey score of each user 10 through the operations of an adder 408 and a multiplier 409. At the same time, the survey score is recorded in the personal record database 402 for follow-up consultation.

Referring to FIG. 4, in one particular embodiment, the intelligent medical resource sharing platform of the present disclosure also includes the calculator 411, which is coupled to the data comparison unit 4042 in the server 40 to calculate the physiological data uploaded by the user 10. As shown in FIG. 4, the calculator 411 includes the adder 408, the multiplier 409, and the value generator 410. The adder 408, the multiplier 409, and the value generator 410 can be connected to each other. The data calculated by the adder 408 and the multiplier 409 can be used to generate the evaluation data through the value generator 410. As shown in FIG. 4, the adder 408 is used to receive the record content and related data of a life state of the user 10 that are transmitted from the personal record database 402 in the server 40 and then processed by the physiological value processing module 404. Further, the related data that is transmitted has been processed numerically, which will be described in the following. At the same time, the adder 408 also receives a numerical content of the physiological state and related data of the user 10 that are transmitted from the physiological value database 401 in the server 40 and then processed by the physiological value processing module 404. The adder 408 is bidirectionally connected to the multiplier 409 to perform numerical operations of Equations 1 to 3, which will be described in the following. A result of the calculation is then transmitted to the value generator 410, so that the result of the calculation is output as a numerical signal, which refers to an output of the calculator 411. The output of the calculator 411 is transmitted to a value comparison and judgment circuit 4043a disposed in the data evaluation unit 4043 of the physiological value processing module 404 for further comparison and judgment, and then the value comparison and judgment circuit 4043a generates an evaluation result and transmits the evaluation result to an evaluation result display generator 4043b, which is used to prepare the evaluation result to be transmitted to a display S. At this time, the value comparison and judgment circuit 4043a also transmits the evaluation result to the medical department recommendation unit 4044. At this time, the recommended medical department output from the medical department recommendation unit 4044 is also transmitted to the evaluation result display generator 4043b, and the recommended medical department for which the user 10 should consult is transmitted to the display S. The display S shown in FIG. 4 refers to a display on the user end device 101. On the other hand, the display S can also be a display on the physician end device 201 or on the pharmacist end device 301.

As shown in FIG. 5, an architecture of an internal circuit module showing the wireless communication coupling between the server 40 and the user end device 101, the physician end device 201, and the pharmacist end device 301 is shown, especially for illustrating internal components correspondingly of the user end device 101, the physician end device 201, and the pharmacist end device 301, and a communication operation firmware of the intelligent medical resource sharing platform of the present disclosure. The user end device 101 of the user 10 can be wirelessly connected to the server 40, and a number of the user end devices 101 can be increased according to a number of participants. The number of the user end devices 101 is not limited if the patient and the relatives each have the user end device 101. The user end device 101 includes a user identity input module 111, a user real-time credentials check reply module 112, and the user physiological data input module 113. The user real-time credentials check reply module 112 is coupled to the user identity input module 111, and can receive and reply to a message transmitted by the user credentials real-time confirmation module 406. The user physiological data input module 113 is coupled to the user real-time credentials check reply module 112, and upload the physiological data of the user 10 to the personal record database 402.

Further, referring to FIG. 3, the physician credentials real-time confirmation module 407 is coupled to the server 40, and is used to perform verification and validate the certification of the physician 20. The physician database 403 is used to store the information of the physician 20, such as the physician's specialty, medical experience, and nationality. Therefore, the physician 20 is not limited to a physician from a specific country, or to traditional Chinese medicine and Western medicine.

As shown in FIG. 5, the physician end device 201 of the physician 20 is wirelessly connected to the server 40, and a number of the physician end devices 201 can be increased according to a number of participants. If physicians from different departments or physicians from a same department but different hospitals attend the consultation, each of the physicians 20 attending the consultation may have the physician end device 201, so that the number of the physician end devices 201 is not limited herein. The physician end device 201 includes a physician credentials input module 211 and a physician real-time credentials check reply module 212. The physician credentials input module 211 provides the physician 20 to perform entry of individual physician credentials. The physician real-time credentials check reply module 212 is coupled to the physician credentials input module 211, and used to receive and reply to a message transmitted by the physician credentials real-time confirmation module 407.

In addition, the server 40 also includes a translation module (not shown in the figures). When the user 10 selects the physician 20 who speaks a different language than the user 10, the translation module can translate the conversation between the physician 20 and the user 10, so that the consultation can be smoothly conducted and the medical resources are no longer limited to a specific area.

As shown in FIG. 3, the physiological value processing module 404 is correspondingly coupled to the physiological value database 401 and the personal record database 402, and configured to process the physiological data transmitted by the physiological value database 401 and the personal record database 402. Accordingly, when the user 10 uploads the physiological data to the personal record database 402, the physiological data of the user 10 is uploaded to the physiological value processing module 404 since the personal record database 402 is coupled to the physiological value processing module 404.

As shown in FIG. 3, the user credentials real-time confirmation module 406 is connected to the server 40, and is used to perform verification and confirm the identity of the user 10. As described above, the physiological value processing module 404 includes the data receiving unit 4041, the data comparison unit 4042, the data evaluation unit 4043, and the medical department recommendation unit 4044. The data receiving unit 4041 is used to receive the physiological data uploaded by the user physiological data input module 113. The data comparison unit 4042 is coupled to the data receiving unit 4041, and is used to compare the data uploaded by the user physiological data input module 113 to the personal record database 402 with the data in the physiological value database 401.

As shown in FIG. 4, the data evaluation unit 4043 is coupled to the data receiving unit 4041. The data evaluation unit 4043 includes the value comparison and judgment circuit 4043a and the evaluation result display generator 4043b. The data evaluation unit 4043 evaluates the physiological state of the user 10 according to the physiological data analyzed by the data comparison unit 4042 to generate the physiological data analysis report 4050 and transmit the same to the display S. The display S can be the display of the user end device 101, the display of the physician end device 201, or the display of the pharmacist end device 301.

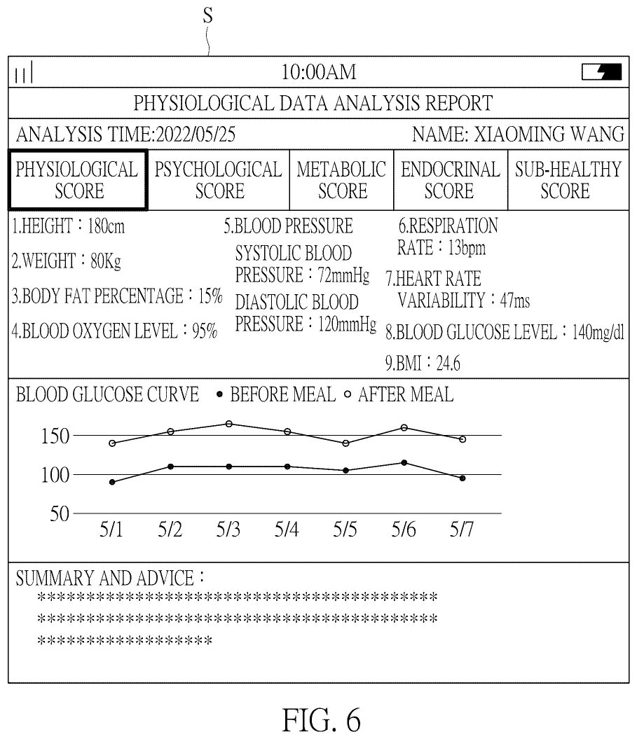

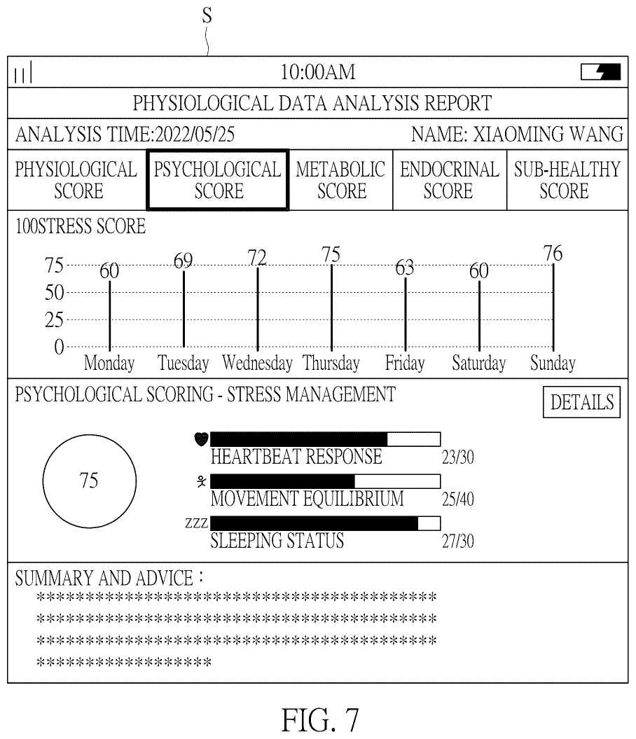

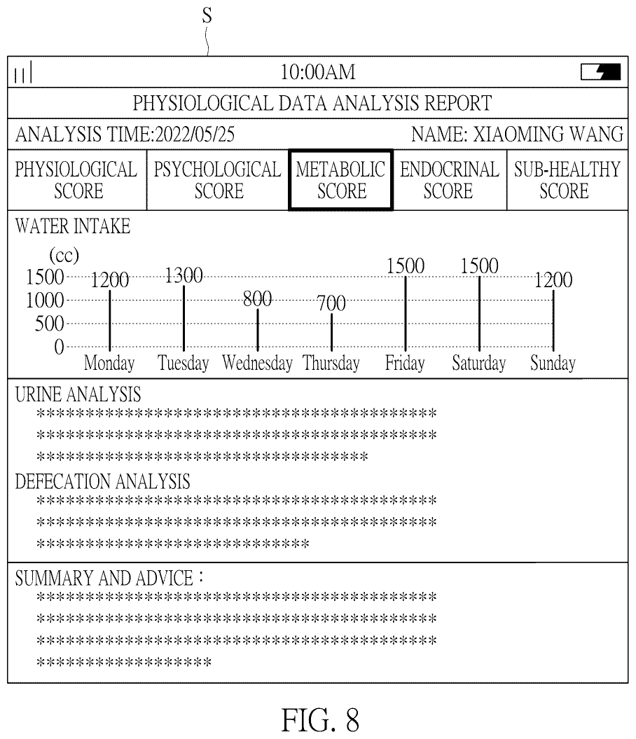

As shown in FIG. 6 to FIG. 8, the physiological data analysis report 4050 includes, but is not limited to, physiological score data, psychological score data, metabolic score data, endocrinal score data, and sub-healthy score data. The psychological score data belong to a psychological group that is used to evaluate a psychological state of the user 10. The metabolic score data belong to a metabolic group that is used to evaluate a metabolic state of the user 10. The sub-healthy score data belong to a sub-healthy group that is used to evaluate a sub-healthy state of the user 10. The endocrinal score data belong to an endocrinal group that is used to evaluate an endocrinal state of the user 10.

In addition, the medical department recommendation unit 4044 is coupled to the data receiving unit 4041, and generates a medical department recommendation according to the physiological data analysis report 4050 and transmits the same to the user end device 101. When the user 10 receives the medical department recommendation on the user end device 101, the user 10 can choose whether or not to make an appointment according to the past medical practice of the user 10, and the user 10 can choose traditional Chinese medicine or Western medicine. In addition, the user 10 can also decide whether to choose traditional Chinese medicine or Western medicine by using a preset judgment equation in the medical department recommendation unit 4044.

The equations for medical choice for the user 10 is as follows:

X=(X1×5)+(X2×3)+(X3×2) (Equation 2);

Y=(Y1×3)+(Y2×2)+Y3 (Equation 3);

where each of X1 and Y1 is a number of traditional Chinese medicine/Western medicine consultations within one month, each of X2 and Y2 is a number of traditional Chinese medicine/Western medicine consultations within two months, and each of X3 and Y3 is a number of traditional Chinese medicine/Western medicine consultations within three months.

If a result of the equations for medical choice calculated by the calculator 411 is X>Y, the user 10 is recommended to consult practitioners of traditional Chinese medicine; if the result of the equations for medical choice is X<Y, the user 10 is recommended to consult physicians of Western medicine; if the result of the equations for medical choice is X=Y, then a questionnaire is provided to judge a mood of the user 10. If the user 10 is in a good mood, the user 10 is recommended to consult the physicians of Western medicine, and if the user 10 is in a depressed mood, the user 10 is recommended to consult the practitioners of traditional Chinese medicine. The aforementioned description is merely an example of the present disclosure, and different platform developers can adjust the judgment methods according to needs of traditional Chinese medicine or Western medicine. That is, the aforementioned implementation pattern can be adjusted according to the needs of developer.

When the user 10 has confirmed the appointment, the physiological data analysis report 4050 is transmitted to the physician end device 201, so that the physician 20 can understand the current health and physiological state of the user 10. In addition, through the consultation platform 500, the physician 20 and the user 10 are able to consult with each other to better understand current symptoms and health state of the user 10. When the consultation is completed, the physician 20 is able to transmit a consult report, a medical record, a life advice, and a prescription to the user end device 101 and the pharmacist end device 301, correspondingly.

The pharmacist credentials real-time confirmation module 405 is coupled to the server 40, and is used to perform verification and validate the certification of the pharmacist 30. Accordingly, the pharmacist end device 301 of the pharmacist 30 can be coupled to the server 40, and the pharmacist end device 301 includes a pharmacist credentials input module 3011 and a prescription receiving module 3012. Therefore, the pharmacist credentials input module 3011 is able to transmit the pharmacist credentials to the pharmacist credentials real-time confirmation module 405 to confirm the identity of the pharmacist 30, and the prescription receiving module 3012 is able to receive the prescription generated by the physician end device 201, so that the pharmacist is able to perform the subsequent dispensing operation. After the pharmacist completes the dispensing procedure, the medicine can be delivered to the user 10 by home delivery. Alternatively, the pharmacist 30 may be located in a pharmacy near a location of the user 10, and the user 10 may pick up the medicine at the pharmacy after the dispensing procedure is completed. In addition, the delivery of medicines can also be carried out by drones or self-driving vehicles. In this way, not only labor costs can be effectively reduced, but also high delivery efficiency can be achieved.

The user 10 can improve the current health stat by following the life advice of the physician 20 and using the medicines dispensed by the pharmacist 30.

### Beneficial Effects of the Embodiments

In conclusion, one of the beneficial effects of the present disclosure is that, the intelligent medical resource sharing platform provided by the present disclosure enables consultation between the physician, the pharmacist, and the user through a consultation platform, which is not limited by distance or time. Through generating the physiological data analysis report by the physiological value processing module, an uncertainty of the user in seeking medical consultation can be effectively reduced. The user often goes to the hospital only because of self-doubt. By comparing the physiological data in the physiological value database, the physiological data analysis report is generated, which allows the user to understand his/her health state before consulting the physician. In addition, through the evaluation and recommendation provided by the intelligent medical resource sharing platform of the present disclosure, the user can have a clearer understanding of which medical department should choose, so that a waste of medical resources can be effectively reduced. Further, when the user really needs to consult the physician, a remote consultation with the physician and the pharmacist can be conducted through the consultation platform, so that the consultation can be kept confidential. In addition, the user can avoid a risk of infection outside of the home. Even if the user is located in a remote site, sufficient medical resources are also available.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An intelligent medical resource sharing platform, comprising:
a server, the server including:
a physiological value database;
a personal record database;
a physiological value processing module correspondingly coupled to the physiological value database and the personal record database, and processing physiological data transmitted from the physiological value database and the personal record database;
a physician database;
a user credentials real-time confirmation module coupled to the server and used to perform checks and confirm an identity of a user; and
a physician credentials real-time confirmation module coupled to the server and used to perform checks and validate a certification of a physician;
at least one user end device wirelessly connected to the server, the at least one user end device including:
a user identity input module;
a user real-time credentials check reply module coupled to the user identity input module and used for real-time reception and reply of a message transmitted by the user credentials real-time confirmation module; and
a user physiological data input module coupled to the user real-time credentials check reply module and used to upload the physiological data of the user to the personal record database; and
at least one physician end device wirelessly connected to the server, the at least one physician end device including:
a physician credentials input module providing the physician to perform entry of individual physician credentials; and
a physician real-time credentials check reply module coupled to the physician credentials input module and used for real-time reception and reply of a message transmitted by the physician credentials real-time confirmation module;
wherein the at least one user end device is used by at least one user, and the at least one user uses the intelligent medical resource sharing platform to obtain a medical department recommendation.

2. The intelligent medical resource sharing platform according to claim 1, wherein the physiological value processing module includes:
a data receiving unit used to receive the physiological data uploaded by the user physiological data input module;
a data comparison unit coupled to the data receiving unit and used to compare data uploaded by the user physiological data input module to the personal record database with data in the physiological value database; and
a data evaluation unit coupled to the data receiving unit, wherein the data evaluation unit evaluates a physiological state of the user according to the physiological data analyzed by the data comparison unit so as to generate a physiological data analysis report and transmit the physiological data analysis report to the at least one user end device and the at least one physician end device, correspondingly.

3. The intelligent medical resource sharing platform according to claim 2, wherein the physiological data analysis report includes psychological score data belonging to a psychological group, and the psychological group is used to evaluate a psychological state of the user.

4. The intelligent medical resource sharing platform according to claim 2, wherein the physiological data analysis report includes metabolic score data belonging to a metabolic group, and the metabolic group is used to evaluate a metabolic state of the user.

5. The intelligent medical resource sharing platform according to claim 2, wherein the physiological data analysis report includes endocrinal score data belonging to an endocrinal group, and the endocrinal group is used to evaluate an endocrinal state of the user.

6. The intelligent medical resource sharing platform according to claim 2, wherein the physiological data analysis report includes sub-healthy score data belonging to a sub-healthy group, and the sub-healthy group is used to evaluate a sub-healthy state of the user.

7. The intelligent medical resource sharing platform according to claim 2, wherein the physiological value processing module further includes:
a medical department recommendation unit coupled to the data receiving unit, and the medical department recommendation unit generates the medical department recommendation and transmits the medical department recommendation to the at least one user end device.

8. The intelligent medical resource sharing platform according to claim 1, wherein the server further includes:
a pharmacist credentials real-time confirmation module coupled to the server and used to perform checks and validate a certification of a pharmacist.

9. The intelligent medical resource sharing platform according to claim 8, further comprising:
a pharmacist end device coupled to the server, the pharmacist end device including:
a pharmacist credentials input module; and
a prescription receiving module;
wherein the pharmacist credentials input module transmits a pharmacist credentials to the pharmacist credentials real-time confirmation module, and the prescription receiving module receives a prescription generated by the at least one physician end device.

10. The intelligent medical resource sharing platform according to claim 1, wherein the physiological value database includes a psychological physiological data group, a metabolic physiological data group, a traditional Chinese medicine physiological data group, and a sub-healthy physiological data group.

11. The intelligent medical resource sharing platform according to claim 1, further comprising:
a calculator disposed in the server and coupled to the physiological value processing module, wherein the calculator is used to calculate the physiological data uploaded by the user and generate evaluation data.

12. The intelligent medical resource sharing platform according to claim 1, further comprising:
a consultation platform correspondingly coupled to the server, the at least one user end device, and the at least one physician end device.
